(19) United States
(12) Patent Application Publication
(10) Pub. No.: US 2003/0172941 A1
(43) Pub. Date: Sep. 18, 2003

(54) Device for the sealing and lubrication of instruments running in trocar sleeves

(76) Inventors: Wolfgang Streifinger, Friedberg-Satzling (DE); Gerald Storm, Augsburg (DE)

Correspondence Address: ST. ONGE STEWARD JOHNSTON & REENS, LLC, 986 BEDFORD STREET, STAMFORD, CT, 06905-5619, US

(21) Appl. No.: 10/379,142

(22) Filed: Mar. 4, 2003

Related U.S. Application Data
(63) Continuation of application No. PCT/EP01/10190, filed on Sep. 5, 2001.

(30) Foreign Application Priority Data
Sep. 6, 2000 (DE) 200 15 388.9

(51) Int. Cl. A61B 19/00

(52) U.S. Cl. 128/899

(57) ABSTRACT

The invention relates to a device to seal and lubricate instruments inserted into trocar tubes, having a sealing cover that can be connected to fit exactly to the insertion end of the trocar tube and has at least one lining lip. The invention is characterized in that a member of porous or fleece-type material that can be moistened with a lubricating agent is mounted in the sealing cover and engages at least partially with the outer circumference of an introduced instrument.

This application is a continuation of pending International Patent Application No. PCT/EP01/10190 filed Sep. 5, 2001, which designates the United States and claims priority of pending German Application No. 200 15 388, filed Sep. 6, 2000.

### FIELD OF THE INVENTION

The invention relates to a device to seal and lubricate instruments inserted into trocars or trocar tubes as stated in the independent patent claim 1.

In minimally invasive surgery such as operations in abdominal reflection technology, trocar tubes are inserted into the abdominal covering in order to enable a video lens and, for instance, surgical instruments to be introduced into the abdominal cavity through these means of access. The trocar tubes provide hermetic sealing against the abdominal covering and, by means of a sealing cover, against the inserted instruments so that the abdominal cavity can be inflated by means of insufflated CO2 gas. In the space thus produced, surgery can be performed with video monitoring by means of instruments introduced through the trocar tube.

The inserted instruments continuously slide along the trocar tube on the outer lining lip of the sealing cover by means of movements during preparation. The lining lip is normally moistened with a drop of parafin oil so that the instrument slides easily and the lining lip is hermetically sealed on the instrument. If this lining zone becomes dry, friction is increased, which makes preparation difficult. During the operation, drops of oil must constantly be reapplied in order to avoid the aforementioned negative effects. This momentary condition demands a certain degree of attentiveness and effort; this allows it to be eliminated.

U.S. Pat. No. 3,871,358 describes a guiding tube with a cylindrical foam rubber member, saturated with a lubricating agent, inserted in its inlet area.

U.S. Pat. No. 5,941,815 presents a guiding tube for surgical instruments, having at both ends foam rubber lining saturated with lubricating agents.

U.S. Pat. No. 5,535,745 describes a guiding tube for a stylet, in which a kind of lubricating pad is intalled, which is advanced by the stylet.

U.S. Pat. No. 5,334,166 describes a device for lubricating guide wires and catheters, which serves this purpose by using a deformable material saturated with lubricant.

In the guiding and lubricating devices described in the state of the art, the degree of hermetic sealing is often not indicated. This will be improved with the present invention.

The aim of the invention is to propose a device to seal and lubricate instruments inserted into trocars and trocar tubes, which device will be hermetically sealed, easily maintained, and self-lubricating.

The aim is fulfilled through the characteristics indicated in patent claim 1.

In keeping with the invention, at least a second lining lip is installed so that the member is mounted and secured between the lining lips.

The sealing cover therefore has an inserted member of porous or fleece-like material—hereafter generally referred to as sponge—which preferably completely surrounds the external circumference of the inserted instrument. For this purpose the sponge is preferably ring-shaped, with its external diameter adjusted to the sealing cover and with its internal diameter adjusted to the instruments to be inserted.

The inserted instrument now glides effortlessly through the trocar tube, continuously lubricated and secured by the reservoir effect of the sponge which is saturated with lubricating agents

In a preferred embodiment of the invention the porous member is saturated with the lubricating agent and inserted in the sealing cover before use, or else already is on hand ready for use, saturated, for instance encapsulated as a ready-to-use one-time article.

The sealing cover, together with the member saturated with lucricating agent, can preferably be available as a ready-to-use one-time article.

Alternatively, the sealing cover can be designed as re-sterilizable and repeatedly usable, whereas the sponge is applied anew each time.

Another possible configuration of the invention provides that the sealing cover has a lubricating nipple and/or a lubricating a lubricating agent supply line for moistening the already inserted porous member with the lubricating agent. Thus the sponge can be re-saturated several times with the lubricating agent in long operations.

As lubricating agent it is possible to use any appropriate, organically safe, inert lubricating agent, for instance paraffin oil.

The porous member itself also consists, understandably, of a chemically and biologically inert material and is a one-way article, which must be sterilized in familiar manner. Before use the sponge is removed from its sterile packaging and inserted in the intended fit in the sealing cover.

In a preferred embodiment of the invention the sealing cover is configured in several parts, where a first part can be applied to fit exactly to the trocar tube and a second part, which encloses the porous member is connected releasably with the first. This multi-part configuration facilitates handling and the insertion of the sponge.

According to this embodiment, the first and second parts of the sealing cover each have at least one lining lip, where the porous member is mounted and secured between the sealing lips of the parts connected to one another. The inserted instrument glides between the sealing lips by means of the lubricating agent contained in the sponge.

The instrument can also be used to seal and lubricate instruments handled through one-way trocars. These one-way trocars are used only once.

A preferred embodiment of the invention is described below by means of illustrations. The drawings and their description indicate additional characteristics and advantages of the invention.

### BRIEF DESCRIPTION OF DRAWINGS

The illustrations are as follows:

FIG. 1: Lateral view of the device.

FIG. 2: Lateral sectional view through the device.

FIG. 3: Cross-section through the sponge.

FIG. 4: Overhead view of the sponge.

### DETAILED DESCRIPTION OF DRAWINGS

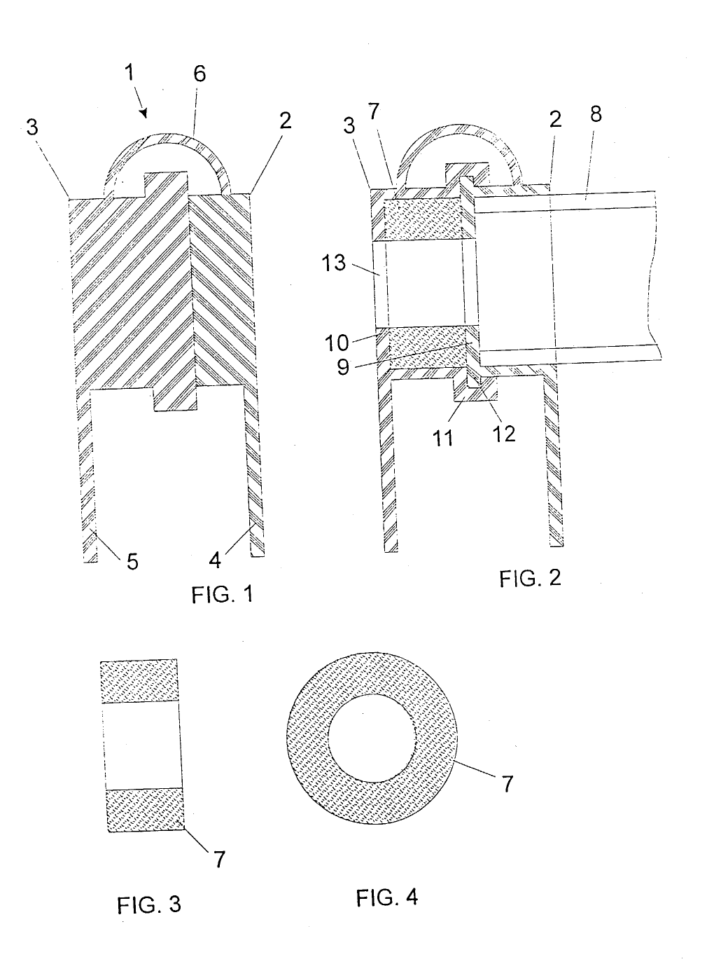

FIG. 1 shows a preferred embodiment of the inventive device. The sealing cover 1 here consists of two parts 2, 3, which preferably are connected non-releasably by means of a connecting belt 6. Each of the parts 2, 3 of the sealing cover includes a securing belt 4 or 5 for better handling of the parts.

As shown in FIG. 2, the first part 2 is configured in such a way that it can be connected to fit exactly to the insertion end of the trocar tube 8. At one end, the part 2 includes a lining lip 9, through whose aperture an operating instrument can be introduced into the trocar tube 8. The lining lip 9 here engages with the outer circumference of the operating instrument. An additional part 3 is connected with the part 2. For this purpose, the part 2 has a surrounding band 12, which can be inserted to fit exactly into an inner groove of the band 12 of the second part 3. In this way both parts are securely but releasably connected together. The second part 3 also has a lining lip 10, through whose aperture 13 the operating instrument can be inserted.

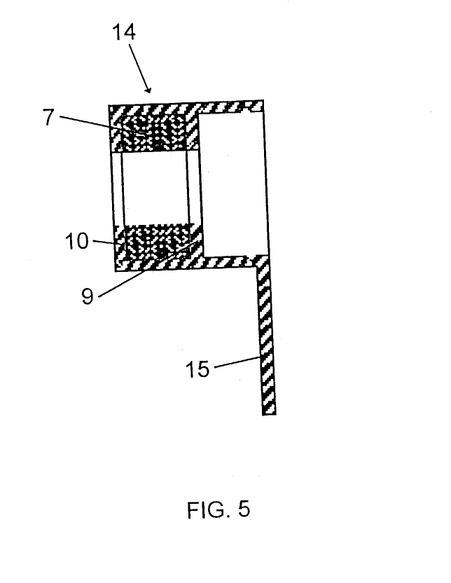

Between the lining lip 9 of part 2 and the lining lip 10 of part 3 a hollow space is configured, in which an absorbable material, preferably a sponge 7, is arranged. As can be seen from FIGS. 3 and 4, the sponge 7 is preferably ring-shaped. The inner diameter of the sponge 7 is thus adjusted to the outer diameter of the operating instrument. Since the sponge 7 happens to be an elastic material, the inner diameter of the sponge 7 can also be smaller than the outer diameter of the operating instrument, since the sponge 7 expands when the instrument is introduced into the aperture 13. The outer diameter of the sponge 7 corresponds to the inner diameter of part 3 of the sealing cover 1.

The sponge 7 is saturated, before the operation, with a lubricating agent, preferably paraffin oil, and is inserted into the fit of the part 3. An instrument introduced into the trocar tube 8 now glides through the aperture 13 along the lining lip 10, along the inner diameter of the sponge 7, and along the lining lip 9, and is thereby moistened with lubricating agent by the sponge 7. This ensures, first, a friction-free gliding of the instrument in the trocar tube 8 and, second, an impeccable tightness between the instrument and the lining lips 9 and 10.

FIG. 5, contrary to FIGS. 1 and 2, shows a one-part sealing cover 14, which preferably is configured as a ready-to-use one-way article. The sealing cover 14 again has two lining lips 9, 10, between which the one-way sponge 7, already saturated with lubricating agent, is inserted. A securing strip 15 provides easier handling of the device.

Figure Key

1 Sealing cover

2 Part

3 Part

4 Securing strip

5 Securing strip

6 Connecting strip

7 Sponge

8 Trocar tube

9 Lining lip

10 Lining lip

11 Band

12 Band

13 Aperture

14 Sealing cover

15 Securing strip

What is claimed is:

1. Device to seal and lubricate instruments inserted into trocar tubes, having a sealing cover (1) that can be connected to fit exactly to the insertion end of the trocar tube (8), in which a member (7) of porous or fleece-type material that can be moistened with a lubricating agent, which at least partially can engage with the outer circumference of an inserted instrument, and with a lining lip (9) arranged in the sealing cover (1), characterized in that in the sealing cover (1) at least one second lining lip (10) is arranged, and the member (7) is mounted and secured between the lining lips (9, 10).

2. Device according to claim 1, characterized in that the sealing cover (14) is a sterile one-way article.

3. Device according to claim 1, characterized in that the sealing cover (1) can be re-sterilized and repeatedly re-used.

4. Device according to any of the preceding claims, characterized in that the sealing cover (1, 14) has a lubricating nipple and/or a lubricating agent supply line for moistening the member (7) with the lubricating agent.

5. Device according to any of the preceding claims, characterized in that the sealing cover (1) is configured in several parts, with a first part (2) that can be affixed to fit exactly on the trocar tube and a second part (3) which receives the porous or fleece-like member (7) and is releasably connected with the first part (2).

6. Device according to any of the preceding claims, characterized in that the first and second parts (2, 3) of the sealing cover (1) each has at least one lining lip (9, 10), where the member (7) is installed and secured between the lining lips (9, 10) of the parts (2, 3) connected to one another.

7. Device according to any of the preceding claims, characterized in that inside the trocar tube (8) an additional porous or fleece-type member, saturated with lubricating agent, is installed.

8. Device according to any of the preceding claims, characterized in that the member (7) is saturated with the lubricating agent.

9. Device according to any of the preceding claims, characterized in that the lubricating agent is an organically safe, inert lubricating agent such as paraffin oil.

10. Device according to any of the preceding claims, characterized in that the member (7) is ring-shaped.

11. Device according to any of the preceding claims, characterized in that the member (7) consists of a chemically and biologically inert material.

12. Device according to any of the preceding claims, characterized in that the member (7) is a sterile one-way article.
